(19) United States
(12) Patent Application Publication
(10) Pub. No.: US 2002/0054850 A1
(43) Pub. Date: May 9, 2002

(54) Monoterpenes and sesquiterpenes as chemotherapeutic sensitizers and radiation sensitizers

(76) Inventors: Michael N. Gould, Madison, WI (US); Steven P. Howard, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 09/878,797

(22) Filed: Jun. 11, 2001

Related U.S. Application Data
(60) Provisional application No. 60/211,506, filed on Jun. 14, 2000.

(51) Int. Cl. A61K 51/00

(52) U.S. Cl. 424/1.11; 424/9.2

(57) ABSTRACT

A method of sensitizing tumor cells to radiation, comprising the step of exposing the tumor cell to an effective amount of at least one monoterpene or sesquiterpene and irradiating the tumor cell, is disclosed.

### CROSS-REFERENCE TO RELATED APPLICATION.

This application claims priority to provisional patent application Ser. No. 60,211,506, filed Jun. 14, 2000, which is incorporated by reference herein.

### BACKGROUND OF THE INVENTION

The incidence of malignant glioma is approximately 12,000 new cases per year. These tumors represent the second leading cause of cancer mortality in people under the age of 35 and the fourth leading cause in those under the age of 54. The exact cause of the disease is unknown, although speculation exists regarding genetic predisposition, chemical, or viral causes. Whatever the cause, recent epidemiological evidence suggests a significant increase in the incidence of these tumors, particularly in the elderly.

The current approach to treatment usually represents a multi-modality approach. Depending upon grade and histopathology, surgery and/or radiation is utilized with or without cytotoxic chemotherapy. For the more advanced types of astrocytoma (Grades III-IV), combined modality approaches have had a questionable impact upon survival; median survival ranges from 40-50 weeks with most patients dead of disease at 2 years.

Despite recent advances in neuro-imaging, neuro-anesthesia, and neuro-surgical techniques, the prognosis of patients with malignant gliomas treated by surgical resection alone remains dismal with a median survival of 4-6 months. This reflects the unique infiltrative growth characteristics of malignant gliomas, which make true “total resection” impossible without causing unacceptable neurologic damage to the patient. To date, radiotherapy has proven to be the most effective treatment for malignant gliomas extending median survival to 8-9 months. Although adjuvant chemotherapy can prolong survival, few patients survive more than 18 months. Furthermore, once patients have tumor progression, conventional chemotherapy has not been shown to prolong survival. There are several reasons why gliomas are relatively resistant to standard chemotherapy including diminished drug delivery to the tumor secondary to the blood-brain barrier, tumor hypoxia, and their relatively low growth fraction. Most importantly, however, is the fact that gliomas tend to have significant intrinsic resistance to most standard cytotoxic agents. Research strategies have been aimed towards the development of new agents directed against novel cellular targets to be used either as single agents or in combination with currently available therapy of proven efficacy.

The development of more effective chemotherapeutic agents intended for combination with radiotherapy such that tumor cell kill is increased while maintaining or improving the therapeutic index has been the clinical rationale for the development of radiosensitizers. Desirable characteristics of clinically useful radiosensitizing agents would include a lack of systemic toxicity and selectivity towards the tumor cell population. Theoretically, the development of an effective radiosensitizer should be particularly appropriate in the management of malignant glioma as 90% of these patients will ultimately develop recurrences within a 2-cm margin of the original tumor, suggesting that these tumors are resistant to standard treatment doses of radiotherapy. In an effort to improve the survival of patients with malignant glioma, numerous clinical studies, both in the single institution and in the larger cooperative group setting evaluating potential radiosensitizers have been conducted. These agents have included halogenated pyrimidine analogs such bromodeoxyuridine and iododeoxyuridine, hypoxic cell sensitizers such as misonidazole and etanidazole, cytotoxic chemotherapeutic agents such as nitrosoureas, cisplatin, carboplatin and taxol, topoisomerase I inhibitors such as topotecan, inhibitors of protein kinase C such as tamoxifen and biological agents such β-interferon. In spite of this large clinical and laboratory effort to identify effective radiosensitizing agents, the overall survival of patients with malignant glioma has remained unchanged over the last two decades. In addition, one of the key drawbacks of most of these agents is their overwhelming systemic toxicity that often limits their clinical usefulness.

Perillyl alcohol (POH) is a monocyclic monoterpene. Monoterpenes are commonly and primarily produced by plants and are found in many commonly consumed fruits and vegetables, including citrus fruits and food flavoring such as mint. Monoterpenes occur in monocyclic, bicyclic, and acyclic forms and are either simple or modified hydrocarbons. We have demonstrated that sesquiterpenes have activities similar to the monoterpenes. We envision that they act with a similar mechanism.

The potential anticancer activity of limonene was first reported in 1971 by Homburger, et al. who observed that limonene, when co-administered with the carcinogen benzo-(rst)-pentaphene, resulted in inhibition of tumor development. These data were extended by Haag, et al. who demonstrated that limonene could cause regression of advanced rat mammary carcinomas.

Following in vitro screening, the naturally occurring hydroxylated monocyclic monoterpene POH was chosen for in vivo testing. Dietary POH was greater than five times more potent than limonene at inducing tumor regression. Dietary administration of POH caused 84% regression of rat mammary carcinoma induced by DMBA and 60% regression of rat mammary carcinoma induced by NMU. Limonene and POH are rapidly metabolized in the rat. Rats given a 2% POH diet for 10 weeks had plasma levels of terpene metabolites of 0.82 mM, while those fed a 10% limonene diet for 10 weeks had plasma levels of 0.27 mM. Thus, the difference in potency between limonene and POH may be due to differences in pharmacokinetics. The observed preclinical antitumor effect of perillyl alcohol has not been limited to mammary carcinoma. The laboratory of P. Crowell has observed an antitumor effect of POH in pancreatic carcinoma models. Only the POH fed hamsters had either regression or no growth of the tumors while control animals showed tumor growth.

The exact mechanism of the antitumor activity of POH has not been established but several potentially important drug-related activities have been observed including: (1) G1 cell cycle arrest and induction of apoptosis; (2) Limonene and POH have been shown to inhibit isoprenylation of a class of 21-26 kD proteins, including small GTP-binding proteins involved in signal transduction, in a dose dependent manner at a point in the mevalonic acid pathway distal to 3-hydroxy-3-methylglutaryl coenzyme A reductase; and (3) Differential gene regulation including overexpression of the mannose-6-phosphate/insulin-like growth factor II (M6P/IGF II) and transforming growth factor-β (TGF-β) type II receptor genes.

Control and regressing POH-treated mammary carcinomas were examined by immunohistochemical methods and demonstrated increases in levels of both the M6P/IGF II receptor, as well as TGF-β, in treated regressing tumors compared with controls. Consistent with the potential importance of the M6P/IGF II receptor in POH-induced tumor regression, responding tumors had increased M6P/IGF II receptor levels compared to those of treated non-responding tumors. Liver tumors from POH-treated animals showed increased mRNA levels for the M6P/IGF II receptor and for the TGF-β type I II, and III receptors compared with those of untreated animals. M6P/IGF-II also enhances the activation of TGF-β that acts as a mammary carcinoma mitogenic inhibitor and differentiating factor. In addition, POH inhibits the isoprenylation of small G proteins including ras-p21 that makes association of these proteins with the plasma membrane impossible and thereby inhibits cellular transformation.

### BRIEF SUMMARY OF THE INVENTION

In one embodiment, the present invention is a method of sensitizing tumor or carcinoma cells, preferably malignant glioma cells, to radiation, comprising the steps of exposing a tumor cell to an effective amount of at least one monoterpene or sesquiterpene, most preferably perillyl alcohol, and irradiating the tumor cell. The tumor cell will thus be more sensitive to the irradiation than a control cell that has not been exposed to the monocyclic monoterpene or sesquiterpene. In another embodiment of the present invention, one sensitizes tumor cells to chemotherapeutics by exposing the tumor cell to an effective amount of at least one monoterpene or sesquiterpene and exposes the tumor cell to the chemotherapeutic.

In a most preferred form of the present invention, the tumor cell is a malignant glioma cell and the cell is exposed to perillyl alcohol before and during irradiation.

It is an object of the present invention to enhance radiation therapy of tumor cells.

It is another object of the present invention to enhance radiation therapy to glioma cells.

Others objects, advantages, and features of the present invention will become apparent to one of skill in the art after review of the specification, claims and drawings.

### BRIEF DESCRIPTION OF THE SEVERAL VIEWS OF THE DRAWINGS

FIG. 1 is a graph of the surviving fraction of T98G glial cells versus dose of POH.

FIG. 2 is a graph demonstrating the effect of perillyl alcohol and radiation on PC3 cells.

FIG. 3 is a graph demonstrating the effect of perillyl alcohol and radiation on DU145 cells.

FIG. 4 is a graph demonstrating the effect of perillyl alcohol and radiation on C6 cells.

FIG. 5 is a graph demonstrating the lack of radiosensitization in M059K cells.

FIG. 6 is a graph demonstrating the effect of perillyl alcohol and radiation on U251 cells.

FIG. 7 is a graph demonstrating the effect of perillyl alcohol and radiation on T98G cells.

FIG. 8 is a graph demonstrating the effect of limonene and radiation on T98G cells.

FIG. 9 is a graph demonstrating the effect of carvonel and radiation on T98G cells.

FIG. 10 is a graph demonstrating the effect of menthol and radiation on T98G cells.

FIG. 11 is a graph demonstrating the effect of citral and radiation on T98G cells.

FIG. 12 is a graph demonstrating the effect of tigllate and radiation on T98G cells.

FIG. 13 is a graph demonstrating the effect of myrecene and radiation on T98G cells.

FIG. 14 is a graph demonstrating the effect of monterpenes on glioblastoma cell line T98G.

### DETAILED DESCRIPTION OF THE INVENTION

In both clinical situations and in laboratory models, tumor cells have been shown to become resistant to cytotoxic agents. At least in some instances, this is the direct result of alterations within apoptotic pathways such that the normal cellular signals that would ultimately result in programmed cell death are no longer intact. We noted that one of the well-characterized actions of POH is the induction of apoptosis and, at least in some model systems, this effect appears to be tumor cell specific. We therefore hypothesize that the pretreatment of resistant glial cell lines with relatively non-toxic doses of POH would predispose these cells to undergo an apoptotic death after treatment with another cytotoxic agent such as radiation. This hypothesis was the basis of our preliminary experiments. The promising in vivo efficacy of POH, with low toxicity, makes it a promising novel agent to be used in combination with other agents of proven efficacy in the treatment of malignant glioma. This information is potentially important as it could generate insights that would guide the development of both new and novel chemotherapeutic agents and more effective combinations of currently available agents for cancer therapy.

In one embodiment, the present invention is a method of sensitizing tumor or carcinoma cells, such as malignant glioma cells, to radiation comprising the step of exposing the cells to an effective concentration of a monoterpene or sesquiterpene, preferably perillyl alcohol. Other preferred cell lines include prostate, colon and pancreatic cancer cells. The preferred effective dose is the maximum tolerated dose, preferably 5-15 grams per day. Treatment is preferably before and during irradiation. The monoterpene or sesquiterpene is preferably administered orally and continuously beginning one week prior to the initiation of radiotherapy and continued for two weeks after the completion of radiotherapy. In addition, radiotherapy is preferably scheduled such that one of the daily doses is administered 1-2 hours prior to daily radiotherapy.

The patient is irradiated in a procedure comparable to current tumor irradiation.

Preferred monoterpenes include perillyl alcohol, limonene, carvone, citral, myrecene and geranyl tigllate. Less preferred monoterpenes include menthol.

### EXAMPLES

### Example 1

### T98G Cell Line and POH

Cell Line:

The T98G cell line was derived from a resection specimen obtained from a patient with glioblastoma multiforme. T98G cells express endogenous mutant p53 and introduction of wild-type p53 sensitizes these cells to radiation. The radioresistance of this cell line has also been attributed to the presence of high intracellular levels of GSH, which down-regulates NF_{K}B binding activity after exposure to ionizing radiation. A clonogenic survival assay was used to assess the response to treatment in vitro, as it is a measure of reproductive capacity.

Clonogenic Survival Assays:

The T98G cell line was obtained from ATCC and maintained in DMEM-F12 medium containing 10% fetal bovine serum, 1% penicillin, streptomycin and 1 mM non-essential amino acids in a humidified incubator at 37° C. Stock solution of POH was made in the medium. One set of plates was treated with graded doses of POH (0.1 mM to 1 mM) for a period of 72 hours. Control dishes were treated with medium alone. All cells were irradiated at a dose rate of 7.5Gy/min using a CS-137 irradiator with single doses ranging from 1Gy to 8.5Gy. The range of radiation doses used encompasses that used to treat patients.

Following irradiation, both POH treated and untreated cells were harvested by trypsinization and washed with PBS. Cells were incubated for two weeks after initial plating, fixed with methanol, stained with crystal violet and the number of colonies per dish quantitated. Survival was determined as the ratio of plating efficiencies for each irradiated group to that of the unirradiated control.

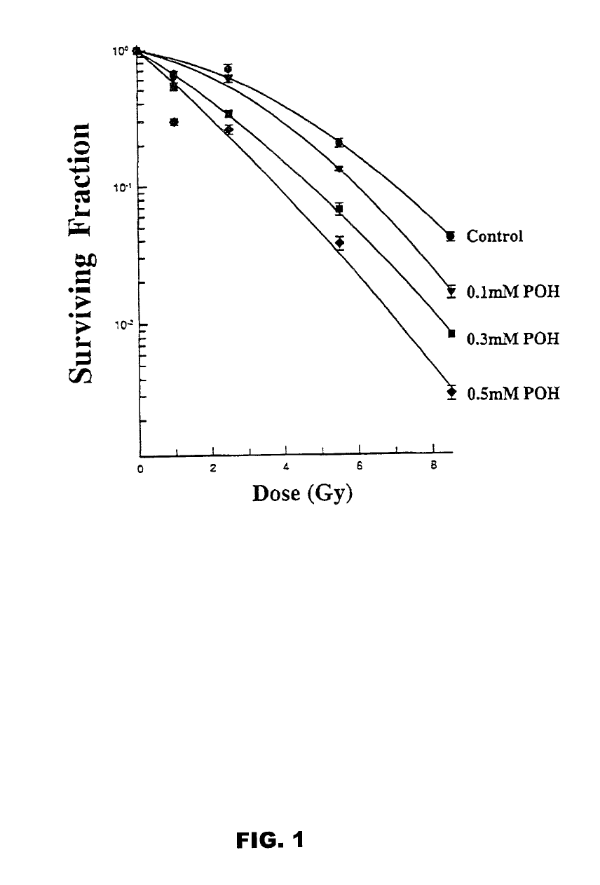

FIG. 1 graphs radiation-dose cell survival curves for control untreated cultures (●) and cultures treated with increasing concentrations of perillyl alcohol; (▾) 0.1 mM, (▪) 0.3 mM, and (♦) before and during irradiation.

Pretreatment of the %98G malignant glioma cell line with perillyl alcohol resulted in a dose dependent sensitization of these cells to radiation induced cell death. This result was observed with concentrations as low as 0.1 mM. Pretreatment of malignant glioma cells with 0.5 mM perillyl alcohol showed the most pronounced effect. Radiosensitization was observed at clinically achievable concentrations of perillyl alcohol.

As shown in FIG. 1, the T98G glial cell line is relatively resistant to cell kill by radiation. Pretreatment of T98G cell line with POH, at minimally cytotoxic concentrations, resulted in a dose dependent sensitization of these cells to radiation induced cell death. This effect was seen (with concentrations as low as 0.1 mM POH) in the clinically relevant dose range of POH. Pretreatment of cells with 0.5 mM POH showed the most pronounced radiosensitization.

### Example 2

### Additional Results with Other Monoterpenes and Cell Lines.

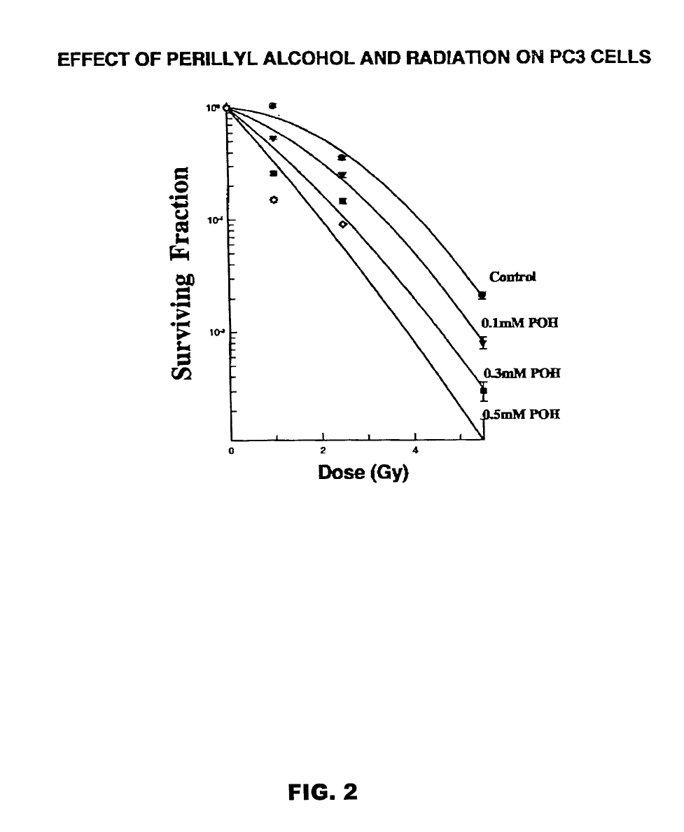

FIGS. 2-14 illustrates additional work with other monoterpenes and cell lines. FIG. 2 demonstrates the effect of perillyl alcohol and radiation on PC3 cells. Subconfluent cultures prostate cancer cell line PC3 were treated with 0.1-0.5 mM of POH for 72 hours and subsequently to increasing doses of radiation from 0.1 Gy to 8.5 Gy. Control cells were treated with the indicated doses of radiation alone. The cells were harvested and an optimum number of cells were allowed to growth for 14 days. The resulting colonies were stained and counted. Each graphed point represents mean values ± SE values of triplicate dishes.

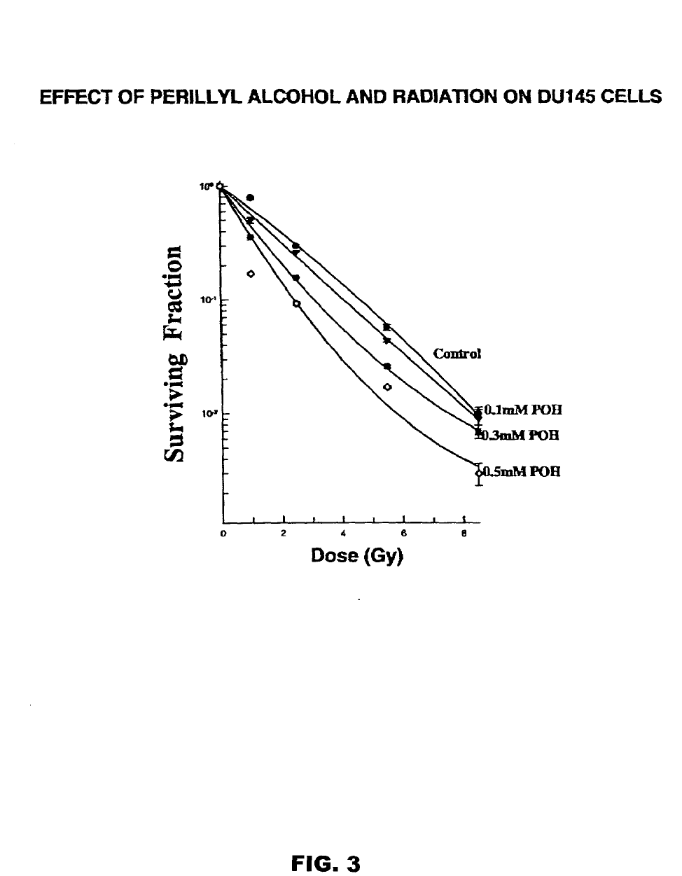

FIG. 3 demonstrates the effect of perillyl alcohol and radiation on DU145 cells. Subconfluent cultures of prostate cancer cell line DU145 were treated with 0.1-0.5 mM of POH for 72 hours and subsequently to increasing doses of radiation from 0.1 Gy to 8.5 Gy. Control cells were treated with the indicated doses of radiation alone. The cells were harvested and an optimum number of cells were allowed to grow for 14 days. The resulting colonies were stained and counted. Each graphed point represents mean values ± SE values of triplicate dishes.

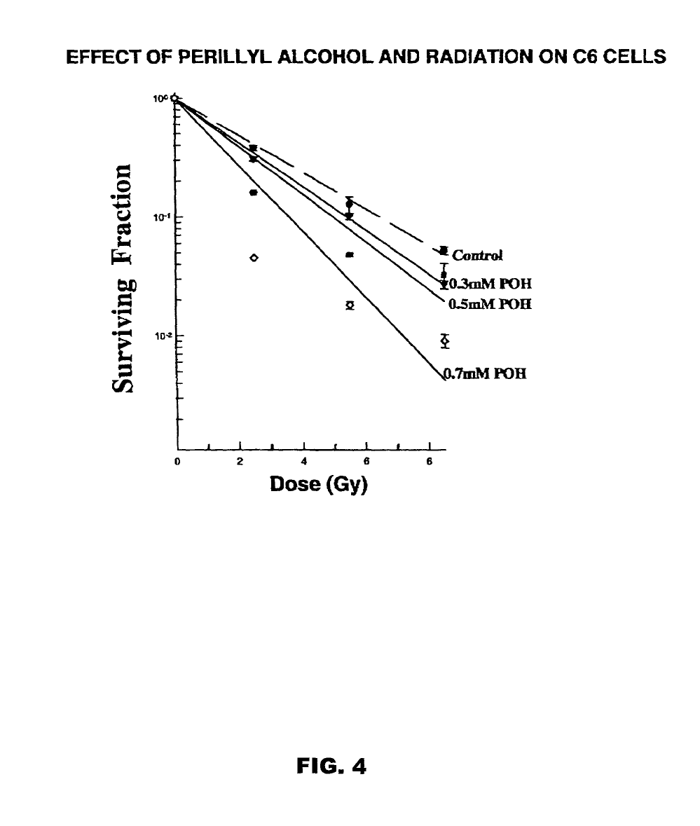

FIG. 4 demonstrates the effect of perillyl alcohol and radiation on C6 cells. Subconfluent cultures of rat glioma cell line C6 were treated with 0.1-0.5 mM of POH for 72 hours and subsequently to increasing doses of radiation from 0.1 Gy to 8.5 Gy. Control cells were treated with the indicated doses of radiation alone. The cells were harvested and an optimum number of cells were allowed to grow for 14 days. The resulting colonies were stained and counted. Each graphed point represents mean values ± SE values of triplicate dishes.

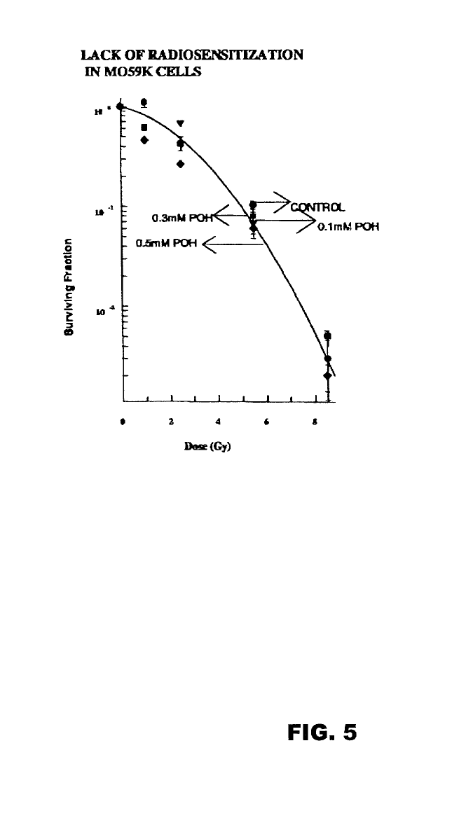

FIG. 5 demonstrates lack of radiosensitization in M059K cells, cells that were not initially radiation resistant. Subconfluent cultures of human glioma cell line M059K were treated with 0.1-0.5 mM of POH for 72 hours and subsequently to increasing doses of radiation from 0.1 Gy to 8.5 Gy. Control cells were treated with the indicated doses of radiation alone. The cells were harvested and an optimum number of cells were allowed to grow for 14 days. The resulting colonies were stained and counted. Each graphed point represents mean values ± SE values of triplicate dishes.

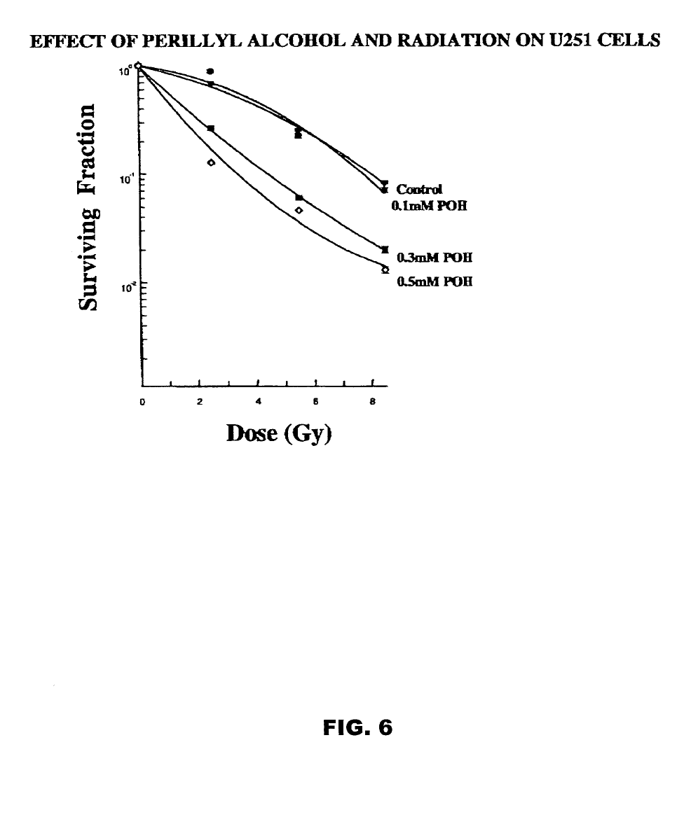

FIG. 6 demonstrates the effect of perillyl alcohol and radiation on U251 cells. Subconfluent cultures of human glioma cell line U251 were treated with 0.1-0.5 mM of POH for 72 hours and subsequently to increasing doses of radiation from 0.1 Gy to 8.5 Gy. Control cells were treated with the indicated doses of radiation alone. The cells were harvested and an optimum number of cells were allowed to grow for 14 days. The resulting colonies were stained and counted. Each graphed point represents mean values ± SE values of triplicate dishes.

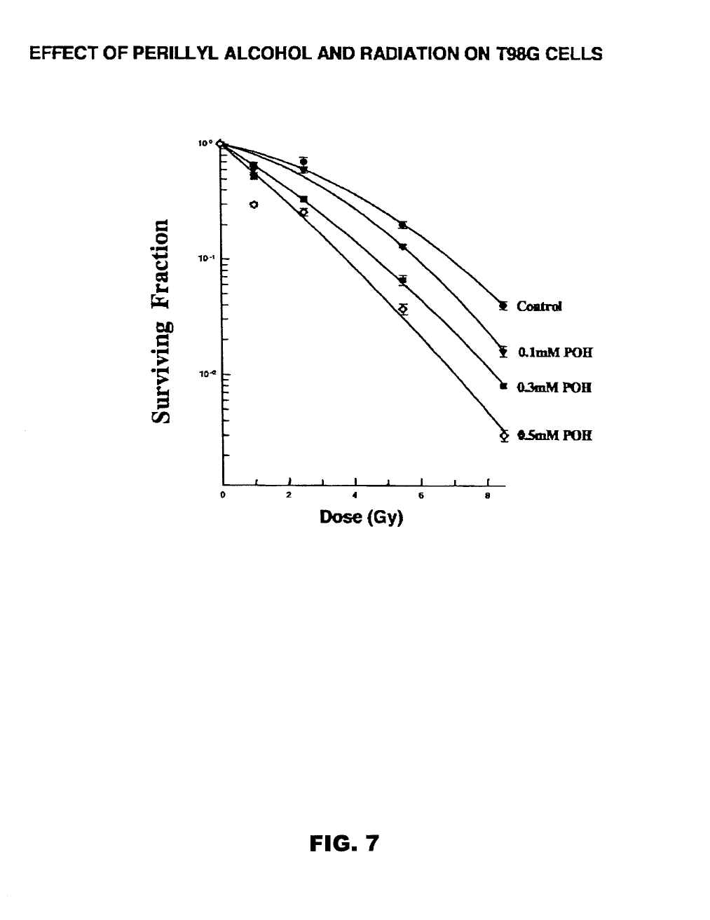

FIG. 7 demonstrates the effect of perillyl alcohol and radiation on T98G cells. Subconfluent cultures of human glioma cell line T98G were treated with 0.1-0.5 mM of Perillyl Alcohol for 72 hours and subsequently to increasing doses of radiation from 0.1 Gy to 8.5 Gy. Control cells were treated with medium alone. The cells were harvested and an optimum number of cells were allowed to grow for 14 days. The resulting colonies were stained and counted. Each graphed point represents mean values ± SE values of triplicate dishes.

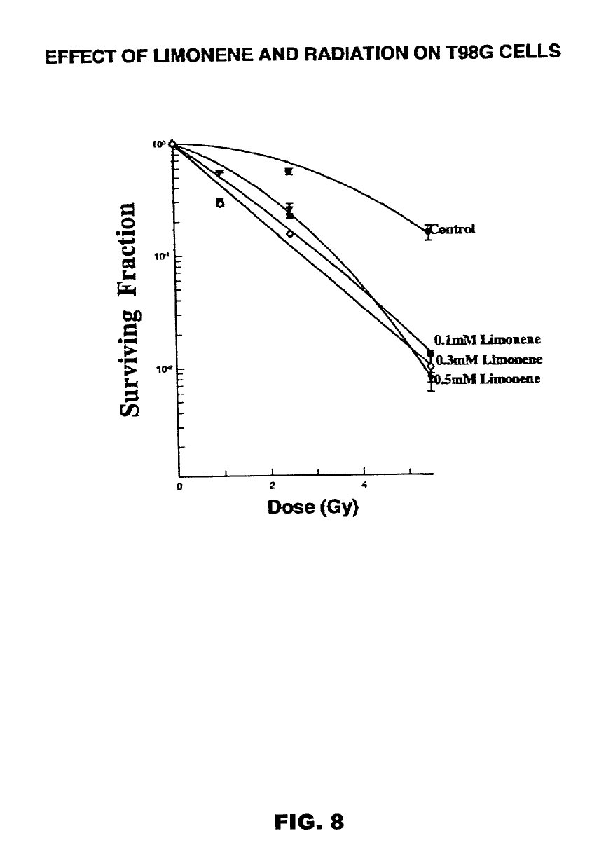

FIG. 8 demonstrates the effect of limonene and radiation on T98G cells. Subconfluent cultures of human glioma cell line T98G were treated with 0.1-0.5 mM of Limonene for 72 hours and subsequently to increasing doses of radiation from 0.1 Gy to 8.5 Gy. Control cells were treated with medium alone. The cells were harvested and an optimum number of cells were allowed to grow for 14 days. The resulting colonies were stained and counted. Each graphed point represents mean values ± SE values of triplicate dishes.

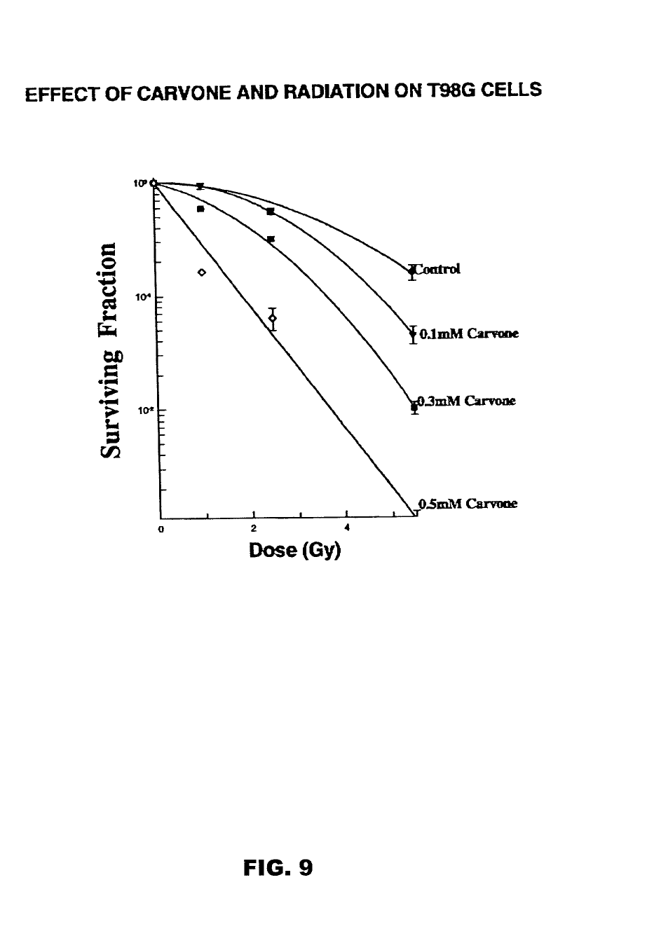

FIG. 9 demonstrates the effect of carvone and radiation of T98G cells. Subconfluent cultures of human glioma cell line T98G, were treated with 0.1-0.5 mM of L-Carvone for 72 hours and subsequently to increasing doses of radiation from 0.1 Gy to 8.5 Gy. Control cells were treated with medium alone. The cells were harvested and an optimum number of cells were allowed to grow for 14 days. The resulting colonies were stained and counted. Each graphed point represents mean values ± SE values of triplicate dishes.

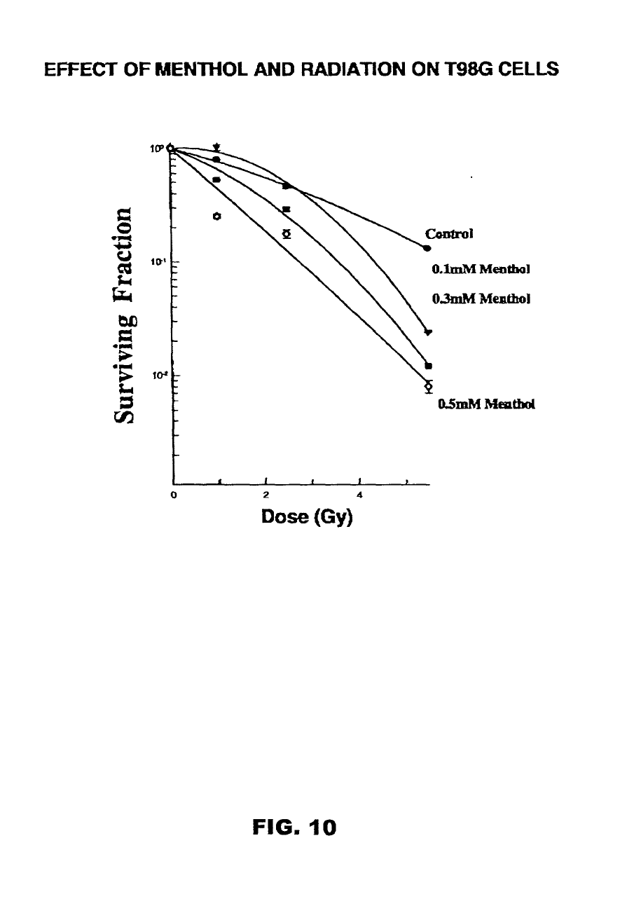

FIG. 10 demonstrates the effect of menthol and radiation on T98G cells. Subconfluent cultures of human glioma cell line T98G were treated with 0.1-0.5 mM of Menthol for 72 hours. Control cells were treated with medium alone. The cells were harvested and an optimum number of cells were allowed to grow for 14 days. The resulting colonies were stained and counted. Each graphed point represents mean values ± SE values of triplicate dishes.

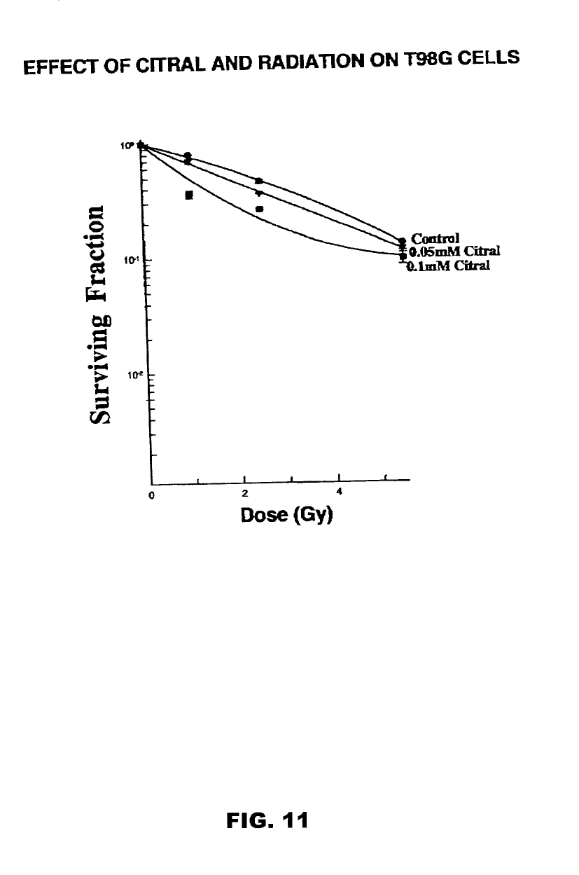

FIG. 11 demonstrates the effect of citral and radiation on T98G cells. Subconfluent cultures of human glioma cell line T98G, were treated with 0.05 and 0.1 mM of Citral for 12 hours. Control cells were treated with medium alone. The cells were harvested and an optimum number of cells were allowed to grow for 14 days. The resulting colonies were stained and counted. Each graphed point represents mean values ± SE values of triplicate dishes.

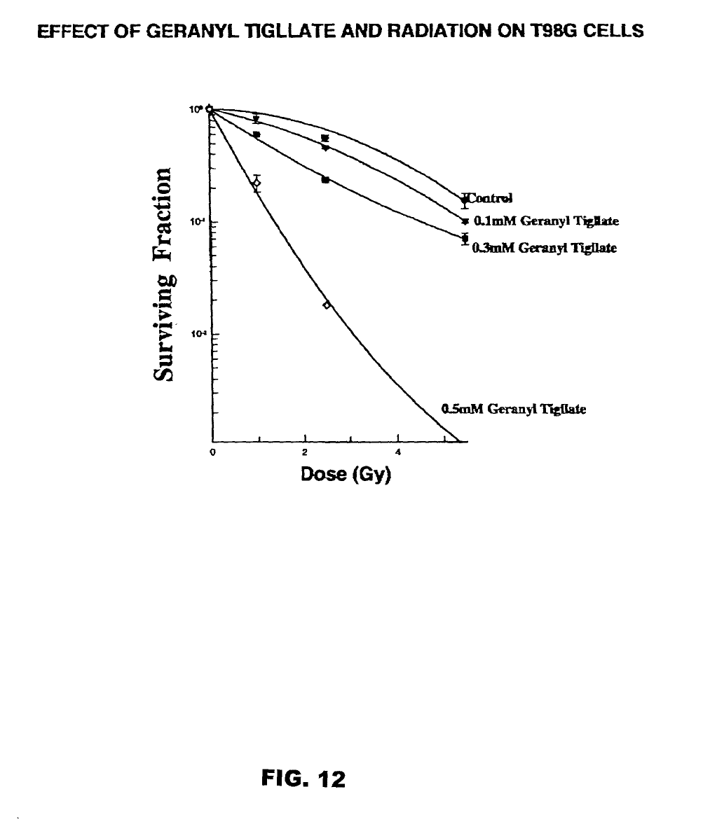

FIG. 12 demonstrates the effect of geranyl tigllate and radiation on T98G cells. Subconfluent cultures of human glioma cell line T98G, were treated with 0.1-0.5 mM of Geranyl Tigllate for 72 hours and subsequently to increasing doses of radiation from 0.1 Gy to 8.5 Gy. Control cells were treated with medium alone. The cells were harvested and an optimum number of cells were allowed to grow for 14 days. The resulting colonies were stained and counted. Each graphed point represents mean values ± SE values of triplicate dishes.

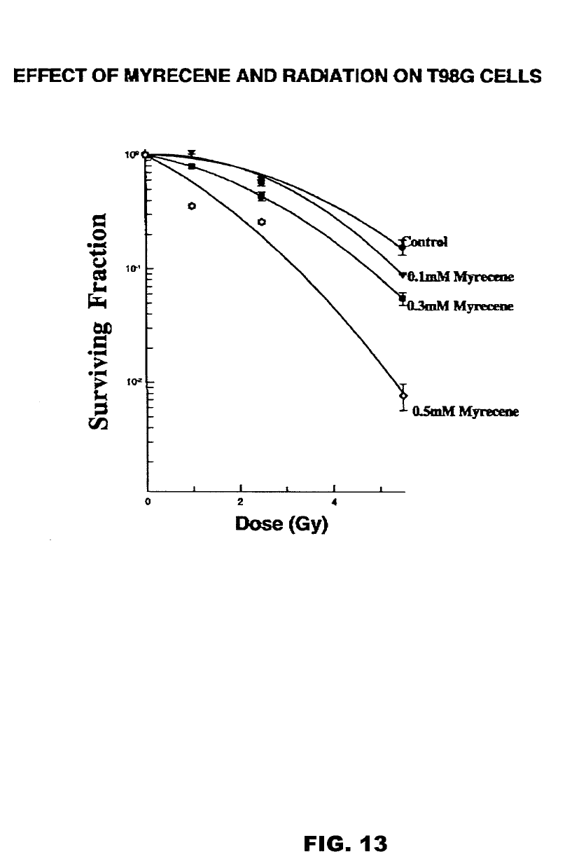

FIG. 13 demonstrates the effect of myrecene and radiation on T98G cells. Subconfluent cultures of human glioma cell line T98G were treated with 0.1-0.5 mM of Myrcene for 72 hours and subsequently to increasing doses of radiation from 0.1 Gy to 8.5 Gy. Control cells were treated with medium alone. The cells were harvested and an optimum number of cells were allowed to grow for 14 days. The resulting colonies were stained and counted. Each graphed point represents mean values ± SE values of triplicate dishes.

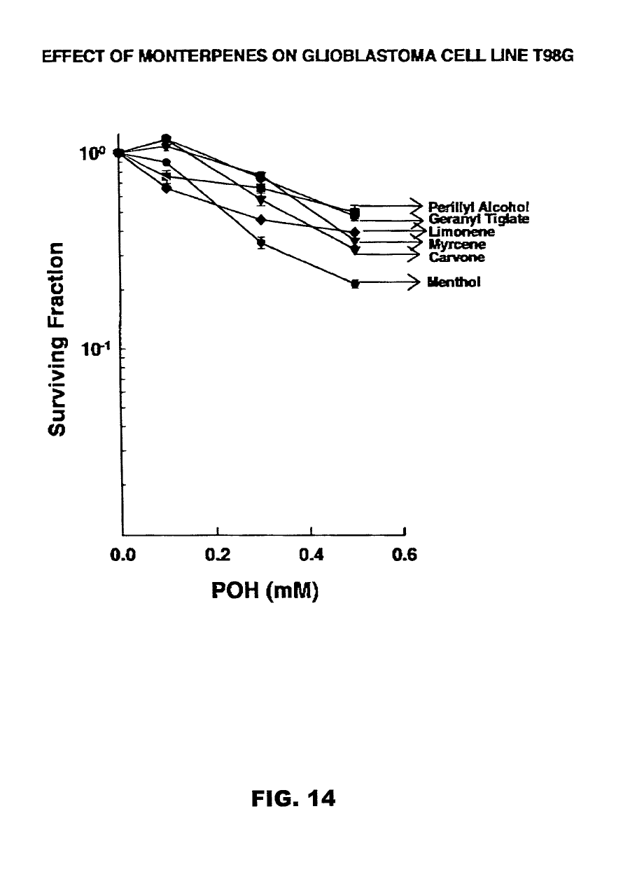

FIG. 14 demonstrates the effect of various monterpenes on glioblastoma cell line T98G. Subconfluent cultures of human glioma cell line T98G were treated with 0.1-0.5 mM of Perillyl Alcohol, Myrcene, L-Carvone, Geranyl Tigllate, Menthol and Limonene for 72 hours. Control cells were treated with medium alone. The cells were harvested and an optimum number of cells were allowed to grow for 14 days. The resulting colonies were stained and counted. Each graphed point represents mean values ± SE values of triplicate dishes.

Table 1 summarizing data from different cell lines treated with radiation, radiation plus POH and POH alone. Subconfluent cultures of human glioma (T98G, M059K, U251), prostate (DU145, PC3), colon (DLD-1, HT29) and pancreatic (MIAPACA) cancer cell lines were treated with 0.1-0.5 mM of POH for 72 hours and subsequently to increasing doses of radiation from 0.1 Gy to 8.5 Gy. Control cells were treated with medium alone. The cells were harvested and an optimum number of cells were allowed to grow for 14 days. The resulting colonies were stained and counted. Each graphed point represents mean values ± SE values of triplicate dishes. Percentage survival was determined as the ratio of plating efficiencies for each irradiated group to that of the unirradiated control.

| | T98G | MO59K | U251 | MIAPACA |
|---|---|---|---|---|
| | % SURVIVAL | % SURVIVAL | % SURVIVAL | % SURVIVAL |
| 5.5 Gy | 0.300 ± 0.009 | 0.111 ± 0.008 | 0.226 ± 0.009 | 0.143 ± 0.296 |
| 0.1 mM POH | 0.755 ± 0.016 | 0.939 ± 0.005 | 1.066 ± 0.200 | 1.503 ± 0.05 |
| 0.3 mM POH | 0.660 ± 0.024 | 0.660 ± 0.020 | 0.397 ± 0.059 | 0.750 ± 0.05 |
| 0.5 mM POH | 0.500 ± 0.006 | 0.623 ± 0.009 | 0.257 ± 0.010 | 0.450 ± 0.07 |
| 0.1 mM POH + 5.5 Gy | 0.130 ± 0.002 | 0.061 ± 0.007 | 0.253 ± 0.004 | 0.154 ± 0.018 |
| 0.3 mM POH + 5.5 Gy | 0.066 ± 0.004 | 0.080 ± 0.015 | 0.046 ± 0.005 | 0.059 ± 0.008 |
| 0.5 mM POH + 5.5 Gy | 0.037 ± 0.014 | 0.056 ± 0.006 | 0.019 ± 0.001 | 0.015 ± 0.0027 |
| | DU145 | PC3 | DLD-1 | HT29 |
| | % SURVIVAL | % SURVIVAL | % SURVIVAL | % SURVIVAL |
| 5.5 Gy | 0.057 ± 0.0035 | 0.011 ± 0.00147 | 0.114 ± 0.015 | 0.013 ± 0.003 |
| 0.1 mM POH | 0.648 ± 0.008 | 0.734 ± 0.038 | 1.690 ± 0.100 | 1.060 ± 0.090 |
| 0.3 mM POH | 0.456 ± 0.0112 | 0.366 ± 0.017 | 0.899 ± 0.070 | 0.714 ± 0.030 |
| 0.5 mM POH | 0.207 ± 0.005 | 0.244 ± 0.009 | 0.019 ± 0.001 | 0.400 ± 0.010 |
| 0.1 mM POH + 5.5 Gy | 0.044 ± 0.00157 | 0.007 ± 0.0009 | 0.038 ± 0.009 | 0.050 ± 0.005 |
| 0.3 mM POH + 5.5 Gy | 0.026 ± 0.00076 | 0.003 ± 0.0058 | 0.050 ± 0.003 | 0.034 ± 0.003 |
| 0.5 mM POH + 5.5 Gy | 0.017 ± 0.005 | 0.001 ± 0.00067 | 0.001 ± 0.001 | 0.006 ± 0.002 |

We claim:

1. A method of sensitizing tumor cells to radiation, comprising the step of exposing the tumor cell to an effective amount of at least one monoterpene or sesquiterpene and irradiating the tumor cell.

2. The method of claim 1 in which the tumor cell is exposed to a monoterpene.

3. The method of claim 2 wherein the monoterpene is perillyl alcohol.

4. The method of claim 2 wherein the monoterpene is selected from the group consisting of perillyl alcohol, limonene, carvone, menthol, citral, myrecene and geranyl tigllate.

5. The method of claim 2 wherein the monoterpene is selected from the group consisting of perillyl alcohol, liminone, carvone, citral, myrecene, and geranyl tigllate.

6. The method of claim 1 wherein the tumor cell is a malignant glioma cell.

7. The method of claim 1 wherein the tumor cell is selected from the group consisting of colon, pancreatic and prostate cells.

8. The method of claim 1 wherein the cell is exposed to the monoterpene or sesquiterpene before and during irradiation.

9. The method of claim 1 wherein the cell is exposed to a monoterpene.

10. A method of sensitizing tumor cells to chemotherapeutic agents, comprising the step of exposing the tumor cell to an effective amount of at least one monoterpene or sesquiterpene and irradiating the tumor cells.

11. The method of claim 10 wherein the monoterpene is perillyl alcohol.

12. The method of claim 10 wherein the monoterpene is selected from the group consisting of perillyl alcohol, limonene, carvone, menthol, citral, myrecene and geranyl tigllate.

13. The method of claim 10 wherein the monoterpene is selected from the group consisting of perillyl alcohol, liminone, carvone, citral, myrecene, and geranyl tigllate.

14. The method of claim 10 wherein the tumor cell is a malignant glioma cell.

15. The method of claim 10 wherein the cell is exposed to the monoterpene or sesquiterpene before or during irradiation.

16. The method of claim 15 wherein the cell is exposed to a monoterpene.
